(19) United States
(12) Plant Patent Application Publication
(10) Pub. No.: US 2003/0056258 P1
(43) Pub. Date: Mar. 20, 2003

(54) VARIETY OF CANNA NAMED "MACTRO"

(76) Inventor: Neil MacCormick, Auckland (NZ)

Correspondence Address: FULWIDER PATTON LEE & UTECHT, LLP, 200 OCEANGATE, SUITE 1550, LONG BEACH, CA, 90802, US

(21) Appl. No.: 09/956,441

(22) Filed: Sep. 18, 2001

(51) Int. Cl. A01H 5/00

(52) U.S. Cl. PLT/263

(57) ABSTRACT

A new and distinct cultivar of canna plant named ‘MACtro’ characterized by a dark to yellow-green leafpattern having multiple linear inter-veinal stripes ranging from a yellow-green coloring which fades to a green-white as the plant matures and a prominent creamy yellow midrib. The leaves offering a vivid contrast to the yellow-orange flowers.

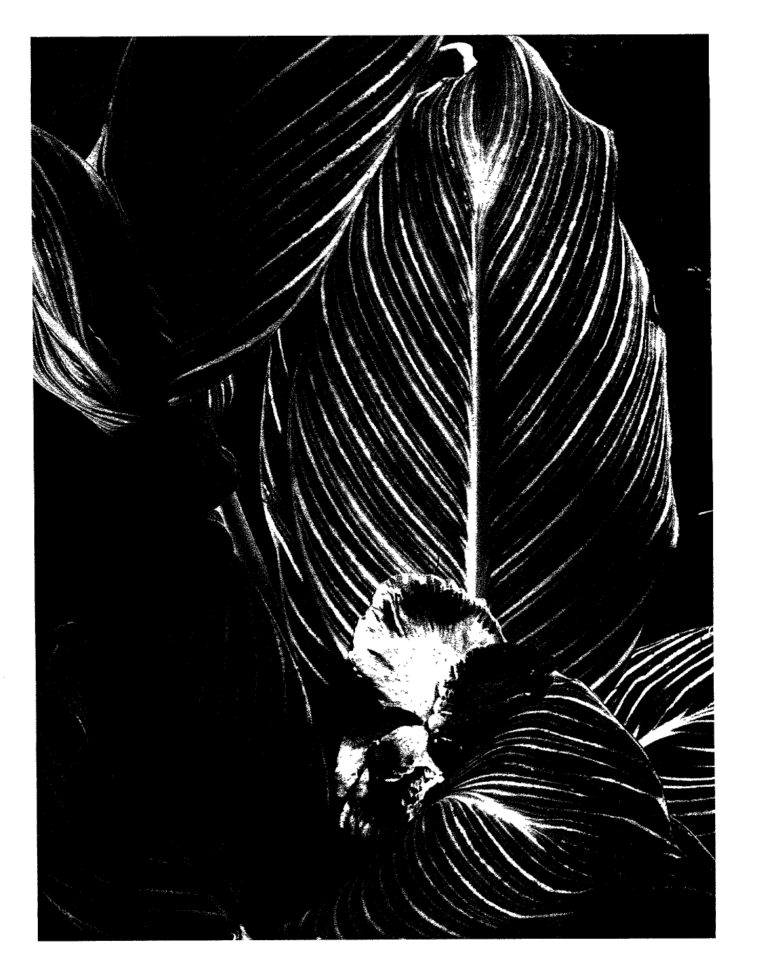

### BACKGROUND OF THE NEW PLANT

The present invention comprises a new and distinct cultivar of canna, botanically known as Canna hybrida and further known by the varietal name ‘MACtro’ (Anthony Tesselaar International No. 011/C). The new variety was discovered in a selective breeding program by Neil MacCormick at Seaview Nurseries Ltd. located at 7 Richardson Road, Alfriston, R.D., Manurewa, Auckland, New Zealand. The new variety is a selection from the open pollenation of Canna ‘Phasion’ (.S. PP10,569) providing both the male and female pollen parents. The new cultivar was selected from the progeny by the inventor Neil MacCormick in Auckland, New Zealand.

The new variety is the result of a selective breeding program taken from selected seedlings in the Nursery at Seaview Nurseries Ltd in the year 2000. These seedlings were selected and propagated through rhizome divisions selecting for differences in color variation of the interveinal striping.

The new cultivar has since been asexually reproduced by rhizome divisions at Seaview Nurseries Ltd. located at 7 Richardson Road, Alfriston, R.D., Manurewa, Auckland, New Zealand and has been repeatedly asexually reproduced by divisions at Seaview Nurseries Ltd. Continued observations from the vegetative divisions have confirmed that the distinguishing features of this new cultivar came true, remain stable and are retained through successive propagation.

The new cultivar was grown in a closed, heated greenhouse at Seaview Nurseries, Manurewa, NZ. It is typically grown under natural light conditions in pots in a bark based potting media. Typical time from division to flowering is 6 months.

### DESCRIPTION OF THE DRAWING

The accompanying drawing illustrates the new cultivar, the color being as nearly true as possible with color illustrations of this type.

### DESCRIPTION OF THE NEW PLANT

The following detailed description sets forth characteristics ofthe new cultivar. The data which defines each characteristic was collected from asexual reproductions grown outdoors, carried out by Seaview Nurseries Ltd in Manurewa, New Zealand. The plant histories were taken on rooted divisions believed to have been potted in a 25 cm pot on approximately November 2000 and flowering in the first growing season on approximately February, 2001 outdoors and color readings were taken in daylight using The Royal Horticultural Society ofLondon Colour Chart, 1995.

### THE PLANT

Classification:

Botanical.—Canna hybrida

Commercial.—‘MACtro’

Parentage.—Seed Parent—Canna hybrid ‘Phasion’Pollen Parent—both male and female are Canna ‘Phasion’

Origin.—Selected seedling

Propagation.—Rhizome division

Rooting habit.—Rhizomes are creeping and fibrous

Form:

Type.—Dense, clump forming, slow growing rhizomatous herb.

Height.—Up to 2000 mm.

Width.—Up to 800 mm.

Growth Rate.—

and Habit.—Upright growth habit; medium in size; growth rate is fast. Grows to full height in single growing season

Strength.—Free standing

Stem.—25 mm in diameter

Leaves: Arranged—spirally.

Shape.—Lamina is large, ovate and broad elliptic, entire and multi-colored, aspect of the leaf is 45 degrees to nearly vertical, venation and midrib are prominent

Length.—Average 400 mm

Width.—Average 220 mm at its widest point

Margin.—Entire

Texture.—Smooth

Color.—Dark grey-green to light yellow-green green with alternating interveinal yellow-green stripes which fade to creamy yellow as the plant matures. Interveinal stripes begin at the midrib of the blade and extend upwardly and outwardly towards the outer edges or tips ofthe leaves, with the shape and color of the stripes being irregular; green leaf varies from Green Group RHS 147 A to Green Group RHS 141 B;

Interveinal.—

Zones.—Yellow-green stripes from Yellow-Green Group RHS 150 C fading to creamy yellow at maturity then Green-White Group RHS 157 C.

Midrib.—Prominent, Yellow-Green Group RHS 150 C

### INFLORESCENCE

General: Inflorescence is a terminal raceme about 300 to 450 mm long, almost branching to second main axis which may be as large as the main axis; main axis always flowers before secondary axis; bracteate, with upper bracts subtending solitary or parred sessile or very short pedicellate. Flowers are bi-sexual, asymmetric, and showy, and are about 130 mm across. Flowering time is May to September—depending on local climatic and environmental conditions. Duration of flowering is typically 2-3 months.

Borne: Terminal raceme about 300-450mm long

Bud: Shape—Lanceolate

Sepal: Quantity—Three in number

Length.—35 mm

Width.—10 mm

Form.—Oval to elliptic, top is rounded off

Petals: Time of flowering early to late with long flowering time

Quantity.—3 unequal united in a basal tube

Size.—Large

Arrangement.—Overlapping

Length.—85 mm

Width.—Up to 20mm wide at widest point

Color

Midzone outside.—Orange Group RHS28 B

Midzone inside.—Orange Group RHS28 B

Margin inside.—Yellow Group RHS6 A

Flowers: Quantity—Many

Size.—Approximately 100 mm

Profile Upper.—Irregular

Profile Lower.—Irregular

Color.—Generally yellow-orange from Orange Group RHS28B with veins having upper surface color closest to RHIS28B, and lower surface color closest to RHS28B; interveinal veins are closest to Yellow-Orange Group RHS17 A

Bract: Color—Violet-Blue Group RHS92A

Fragrance: None detected

Persistence: Disease Resistance—No known specific disease resistence Lasting Quality—Not relevant

### REPRODUCTIVE ORGANS

Stamens: Single stamen is petaloid with solitary marginal anther, united to fleshy-style; staminoides are two in number with one almost as big as petal and same color, recurved; nectarial glands present; ovary inferior, three locular, verrucose, ovules numerous, style petaloid

Filaments—Color—ranging from Orange Group RHS24A to Orange Group RHS25A

Anther: Solitary marginal anther Orange Group RHS28A

Pistils:

Style.—Orange Group RHS28A

Fruit: Size: up to 20 mm, not fully developed, verrucose, capsule

Color.—Immature—Green Group RHS137A Mature—Red-Purple Group RHS178A

Seeds.—Do not develop

### GENERAL CHARACTERISTICS

‘MACtro’ is primarily distinguished from its parent Canna ‘Phasion″ and other hybrid cultivars of Canna genus by the alternatively striped or interveinal coloring of its leaves. More specifically ‘MACtro’ takes the form of a slow growing perennial herb with horizontal subterranean rhizomes with multiple striped or interveinal leaves, and its creamy yellow midrib. The leaf is dark green with interveinal stripes ranging from yellow-green stripes which fade to a creamy yellow as the plant matures. The interveinal stripes of ‘MACtro’ begin at the midrib of each leaf and extend upwardly and outwardly toward the edges or tips of the leaves, with the shape and color of the stripes being irregular. The green leaves and interveinal striping providing a rich background contrast to the yellow-orange flowers. Typically ‘MACtro’ has approximately 8-10 racemes per year of growth.

I claim:

1. A new and distinct variety of Canna plant, substantially as shown and described.
